(19) United States
(12) Patent Application Publication
(10) Pub. No.: US 2017/0043139 A1
(43) Pub. Date: Feb. 16, 2017

(54) FLUID DELIVERY OR REMOVAL SYSTEM

(71) Applicant: Vascular Solutions, Inc., Minneapolis, MN (US)

(72) Inventor: Howard Root, Excelsior, MN (US)

(21) Appl. No.: 15/144,879

(22) Filed: May 3, 2016

Related U.S. Application Data
(60) Provisional application No. 62/203,439, filed on Aug. 11, 2015.

(51) Int. Cl. A61M 25/10

(52) U.S. Cl. CPC A61M 25/10181; A61M 2205/3334; A61M 2230/30

(57) ABSTRACT

Fluid delivery or removal systems and methods for use during a medical procedure are disclosed. A system can include a base member, a receptacle configured to be received by a chamber of the base member, and a pressure sensor. The base member can include a piston having the pressure sensor positioned at or near its tip, a powered actuator for imparting motive forces to the piston, and a rechargeable power supply to energize the actuator. The receptacle is configured to contain a volume of fluid partially retained by a seal member. A surface of the seal member opposite the fluid can include a cavity to receive and engage the piston's tip when the receptacle is placed in the chamber. Based on a user-defined parameter input (e.g., a desired pressure set point) received by the system, the piston can be driven in an axial direction by the powered actuator, thereby urging an amount of fluid through a distal opening in the receptacle.

### CLAIM OF PRIORITY

This non-provisional patent document claims the benefit of priority under 35 U.S.C. §119(e) to U.S. Provisional Patent Application Ser. No. 62/203,439, entitled “FLUID DELIVERY OR REMOVAL SYSTEM” and filed on Aug. 11, 2015, which is herein incorporated by reference in its entirety.

### TECHNICAL FIELD

This patent document relates to medical devices. More particularly, but not by way of limitation, the patent document relates to fluid delivery or removal systems and methods.

### BACKGROUND

Fluid delivery or removal systems and methods are used for a variety of medical procedures. Fluid delivery systems and methods can be used to pump liquid into a body cavity of a patient or a dilatation balloon of a catheter, particularly where it is desirable to limit the volume, pressure or duration of the liquid delivered. In spinal anesthesia procedures, for example, an anesthetic solution is pumped from a fluid delivery system and through a needle into the epidural, subdural and inelastic subarachnoid spaces, which requires careful attention to the volume and pressure of the solution delivered. In various other medical instances, it is similarly desirable to have an indication of the volume of fluid delivered to and the pressure within a blood vessel, a body cavity, or a medical device during a procedure.

Manual fluid delivery or removal systems require an operator to use both hands to control a fluid delivery or removal process. As a result, each time an adjustment in the location of a dilatation balloon in a patient's vessel must be made, for example, the operator must move at least one hand from the fluid delivery or removal system to a tubular body attached to the dilatation balloon to accomplish its relocation. The operator must then return to the delivery or removal system with both hands.

### Overview

The present inventor recognizes that rather than having to use both hands to control a fluid delivery or removal system, it would be desirable for an operator to only use one hand, leaving the second hand free to control the position of a dilatation balloon in a vessel or to perform other needed tasks. The present inventor further recognizes that the fluid delivery or removal system needs to be easy to use, precise in its measurements, able to deliver or remove fluid at a uniform rate, and inexpensive to maintain.

The present fluid delivery or removal systems can include a disposable or refillable receptacle, a reusable base member, and a pressure sensor. The receptacle can be configured to contain a volume of fluid and extend from a first end having an opening of a first diameter to a second end having an opening of a smaller, second diameter. A seal member can be positioned near the first end within the opening of the first diameter and engage an interior surface of the receptacle along its perimeter. The base member can include a piston and a powered actuator for imparting motive forces to the piston. This automated piston movement frees one of the operator's hands during use. A tip of the piston can be engageable with the seal member when the receptacle is placed in a chamber of the base member. Fluid pressure is monitored during movement of the piston by a pressure sensor positioned at or near the tip of the piston. The pressure sensor allows the system to provide accurate control over fluid volume or pressure delivered to a dilatation balloon based on a user-inputted parameter set point, for example.

A method can comprise inserting a receptacle configured to contain a volume of fluid retained in part by a seal member into a chamber of a base member. A tip of a piston housed in the base member can be engaged with the seal member, with a pressure sensor positioned between a portion of the piston and a portion of the seal member. A fluid-tight pathway between a distal opening of the receptacle and a distal end of a catheter can be established, and a desired parameter set point can be inputted using one or more input receiving members accessible from the outside of the base member. The desired parameter set point can cause the piston to advance or retract in an axial direction to move the seal member within the receptacle and fluid through the pathway. A pressure applied to fluid within the pathway can be sensed using the pressure sensor before, during, or after movement of the piston and seal member.

These and other examples and features of the present systems and methods will be set forth, at least in part, in the following Detailed Description. This Overview is intended to provide non-limiting examples of the present subject matter—it is not intended to provide an exclusive or exhaustive explanation. The Detailed Description below is included to provide further information about the present systems and methods.

### BRIEF DESCRIPTION OF THE DRAWINGS

In the drawings, like numerals can be used to describe similar features and components throughout the several views. The drawings illustrate generally, by way of example but not by way of limitation, various system and method embodiments discussed in this patent document.

FIGS. 1A-C illustrate a dilatation balloon of a catheter positioned within and expanding against a lesion of a vessel, as constructed in accordance with at least one embodiment.

FIGS. 2-3 respectively illustrate assembled and exploded views of a fluid delivery or removal system, as constructed in accordance with at least one embodiment.

FIG. 4 illustrates a functional flow diagram of components included in a fluid delivery or removal system, as constructed in accordance with at least one embodiment.

FIGS. 5A-5C illustrates locking configurations between a seal member and the tip of a piston, as constructed in accordance with at least one embodiment.

FIG. 6 illustrates a charging station engageable with a base member, as constructed in accordance with at least one embodiment.

FIG. 7 illustrates a method of using a fluid delivery or removal system, as constructed in accordance with at least one embodiment.

The drawing figures are not necessarily to scale. Certain features and components may be shown exaggerated in scale or in schematic form, and some details may not be shown in the interest of clarity and conciseness.

### DETAILED DESCRIPTION

Although a majority of the following description generally explains and illustrates use of the present systems and methods with reference to inflation or deflation of a dilatation balloon for use in an angioplasty procedure, it should be understood that the systems and methods can also be used for delivering fluid to, or removing fluid from, a blood vessel, a body cavity or another medical device during many kinds of procedures.

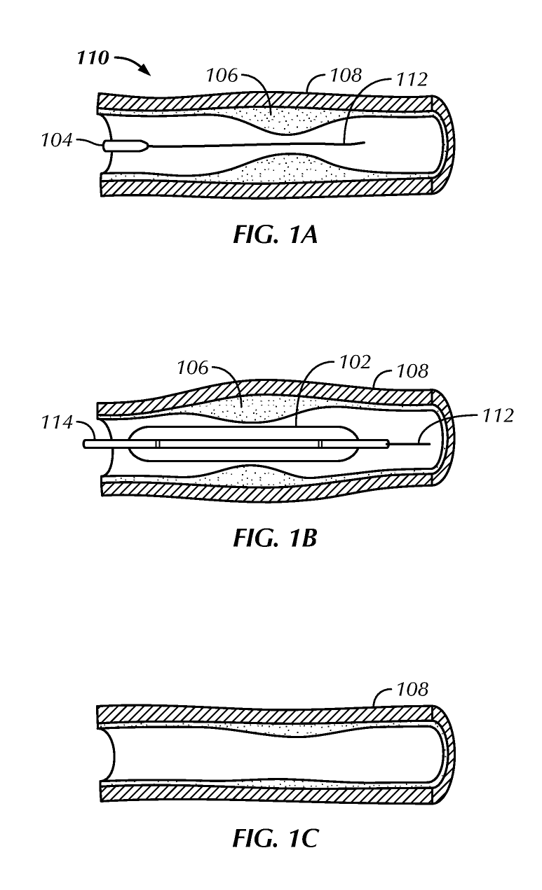

FIGS. 1A-C illustrate a dilatation balloon 102 of a catheter 104 positioned within and expandable against a lesion 106 of a vessel 108. Percutaneous transluminal coronary angioplasty (PTCA) 110 is a surgical procedure used for treating coronary disease and involves the dilatation balloon 102 being inserted through an incision made in a patient's groin or in an artery of the patient's arm and advanced through the vessel by means of a guide catheter and a guidewire 112. The dilatation balloon 102 can be a component of the catheter 104, which can also include a tubular body 114 and a proximal manifold, with the balloon 102 attached to a distal portion of the tubular body 114 and configured to receive inflation fluid from a fluid delivery system.

The dilatation balloon 102 can be advanced until it is located near the middle of the lesion 106. Once located near the middle of the lesion 106, the dilatation balloon 102 can be inflated to a pressure between 10 and 20 atmospheres (atm) for 0 to 60 seconds, for example. The dilatation balloon 102 can then be deflated and the procedure can be repeated a plurality of times, slightly increasing the inflation pressure each time to further compress and reduce vessel blockage of the lesion 106. Once this series of inflations and deflations is completed and the vessel 108 is cleared or its patency is sufficiently opened as shown in FIG. 1C, the dilatation balloon 102 can be removed.

While PTCA is a much less traumatic procedure than coronary artery bypass surgery, controlling the pressure and duration of the inflation periods can be essential to the safety of the patient. When the dilatation balloon 102 is inflated and compressed against the lesion 106, blood flow to the heart is temporarily shut off. This can create a potential for initiating cardiac arrest. Accordingly, the pressure exerted on the vessel 108 by the dilatation balloon 102 and the duration of the blockage created by inflating the balloon should be carefully controlled by the operator. The pressures and duration of the inflations can be based on an assessment of the rigidity of the lesion 106 (e.g., stenosis), the health of the patient and the patient's ability to withstand a temporary stoppage of blood flow to portions of the heart.

The present systems and methods allow for automated inflation or deflation in a self-contained, automated and programmable unit. An operator can input a desired pressure or other parameter set point into the system using one or more input receiving members, and a controller of the system can actuate a powered actuator (e.g., a screw motor), which in turn can impart motive forces to a piston based on a comparison of a sensed fluid pressure within the system and the desired parameter set point. Inflation or deflation pressures can be automatically displayed and recorded for use in future procedures with each patient. In contrast to manual fluid delivery or removal systems that tend to emit or intake fluid sporadically, the present systems and methods allow for uniform, repeatable delivery or removal of fluid. The application of fluid in a uniform manner enables tissues and nerves to gradually adapt to an increase in fluid pressure or volume, which can make treatment safer and more effective.

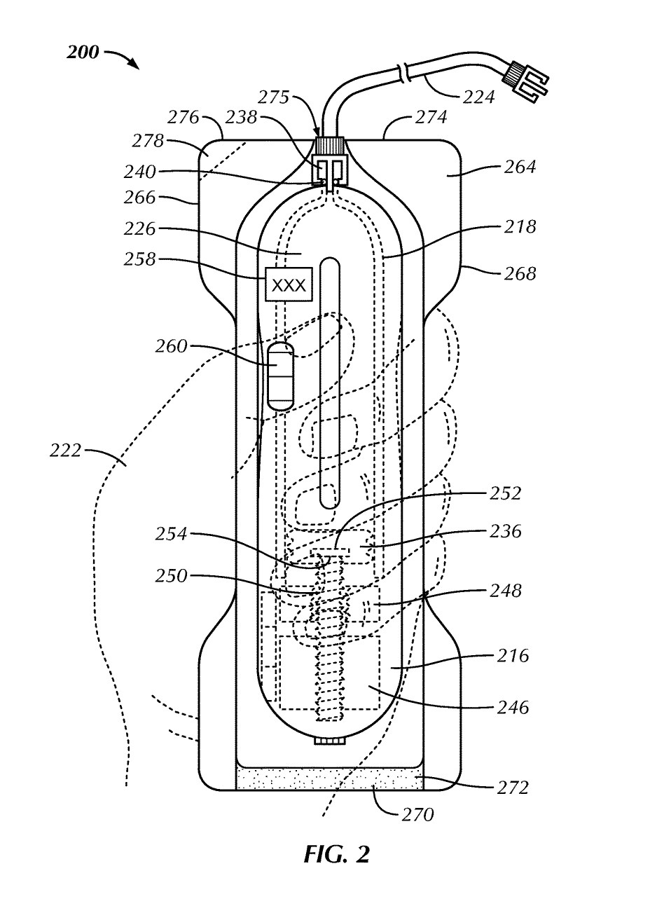

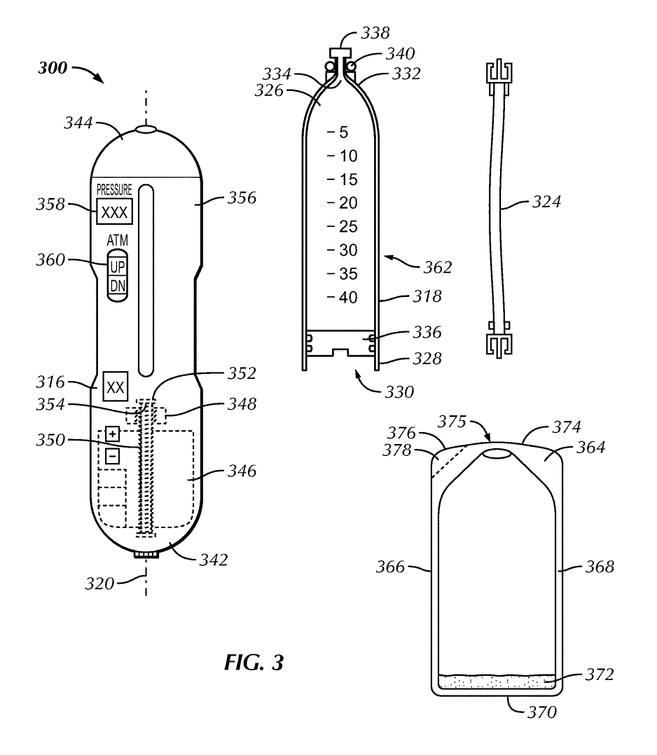

FIGS. 2 and 3 respectively illustrate assembled and exploded views of a fluid delivery or removal system 200, 300 for use with a medical device, such as a dilatation balloon. The system 200, 300 comprises a base member 216, 316, which can be a multi-use component, and a disposable or refillable receptacle 218, 318 detachably mountable to the base member 216, 316 along a central axis 320. When assembled, the system 200, 300 can be held and controlled by a single hand 222 of an operator and used to apply fluid to the dilatation balloon for its inflation or deflate the balloon after it has been inflated for a desired duration. The system 200, 300 can be directly coupled to a catheter including the dilatation balloon or indirectly coupled to the catheter via an extension line 224, 324 that transfers fluid to and from the receptacle 218, 318.

The receptacle 218, 318 can be of a generally cylindrically shape of various sizes and proportions and can be configured to contain a volume of fluid 226, 326, a portion of which can be introduced into the dilatation balloon during its inflation. The receptacle 218, 328 can extend from a first end 328 having an opening 330 of a first diameter to a second end 332 having an opening 334 of a second diameter smaller than the first diameter. A seal member 236, 336 can be positioned near the first end 328 of the receptacle 218, 318 and engaged with its interior surface to partially retain the fluid 226, 326. The second end 332 of the receptacle 218, 318 can have an attachment structure 238, 338 that engages with an end of the dilatation balloon catheter or the extension line 224, 324. In an example, the attachment structure 238, 338 can include a groove adapted to receive an O-ring member 240, 340 to facilitate a fluid tight seal between the receptacle 218, 318 and the extension line 224, 324, which can be engaged via a snap-fit or rotating luer connection.

The base member 216, 316, like the receptacle 218, 318, can have a generally cylindrical shape and extend from a first end 342 to a second end 344. The first end 342 of the base member 216, 316 can house a rechargeable battery 246, 346, a powered actuator 248, 348, a piston 250, 350 and a pressure sensor 252, 352. The piston 250, 350 can have a tip 254, 354 including the pressure sensor 252, 352 and be engageable with the seal member 236, 336 when the receptacle 218, 318 is received by the base member 216, 316. The powered actuator 248, 348, which is energized by the rechargeable power 246, 346, can impart axial motive forces to the piston 250, 350. In the example illustrated, the powered actuator 248, 348 includes a screw motor that can engage threads extending around a surface of the piston 250, 350. The piston 250, 350 can move in an axial direction in response to motive forces applied by the screw motor. The second end 344 of the base member 216, 316 can form a chamber 356 sized and shaped to receive at least a portion of the receptacle 218, 318. An outer surface portion of the base member 216, 316 can include a display 258, 358 and one or more input receiving members 260, 360.

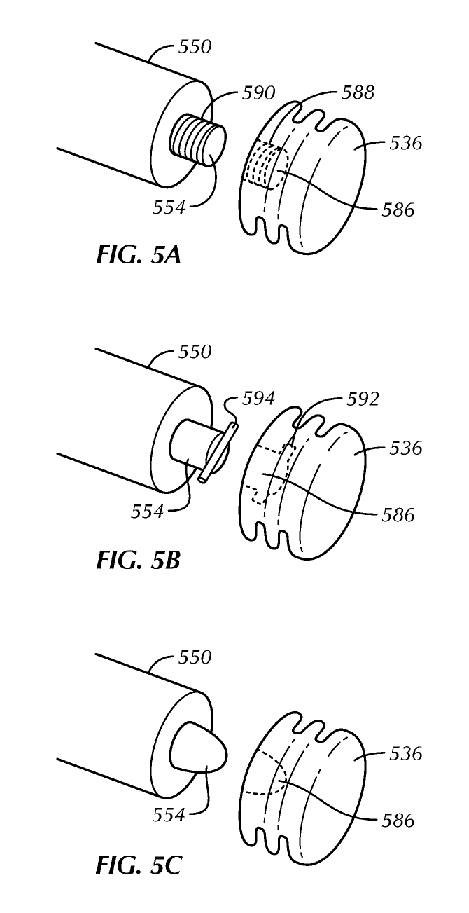

The seal member 236, 336 can be displaceable within the receptacle 218, 318 through axial movement of the piston 250, 350, thereby altering the volume of fluid 226, 326 within the receptacle 218, 318. Moving the seal member 236, 336 distally (i.e., further into the receptacle) can decrease the volume of fluid 226, 326 in the receptacle 218, 318 and increase the pressure in the closed system 200, 300. Moving the seal member 236, 336 proximally can increase the volume of fluid 226, 326 in the receptacle 218, 318 and decrease the pressure in the closed system 200, 300. The seal member 236, 336 can be composed of a soft rubber bulb that engages the interior of the receptacle 218, 318 in a fluid-tight manner such that by moving the piston 250, 350 distally, positive pressure exerted on the fluid 226, 326 contained within the receptacle 218, 318 and any extension line 224, 324 tubing attached to the receptacle 218, M8 can be applied to the dilatation balloon. The seal member 236, 336 can be secured to the tip 254, 354 of the piston 250, 350 in a variety of ways, some examples of which are disclosed in association with FIGS. 5A-5C, below.

Before performing a fluid transfer procedure, the receptacle 218, 318 can be mounted to the base member 216, 316 such that the tip 254, 354 of the piston 250, 350 is engaged with the seal member 236, 336. The base member 216, 316 can be configured to receive the receptacle 218, 318 via its chamber 356. The receptacle 218, 318 and the seal member 236, 336 can be in the form of a ‘ready to use’ kit 362, where the receptacle contains fluid 226, 326. In this case, no preparation is necessary other than connecting the kit 362 to the base member 216, 316. In some examples, the kit 362 is disposable after use and the receptacle 218, 318 is made of a relatively inexpensive medical grade plastic such as polypropylene, polyethylene or polycarbonate. In other examples, the kit 362 can be sanitized and refilled with the same or a different fluid 226, 326.

The second end 344 of the base member 216, 316 forming the chamber 356 can surround a portion of the receptacle 218, 318 to restrain its outward expansion during a fluid delivery procedure. At least the second end 344 of the base member 216, 316 can be made of a material capable of restraining the outward expansion of the receptacle 218, 318 during a fluid delivery procedure, as the material of the receptacle 218, 318 may not alone be capable of withstanding the high pressures associated with certain fluid delivery procedures, such as those involved in angiography. In these situations, the second end 344 of the base member 216, 316 can be used to limit the radial expansion of the receptacle 218, 318 that may otherwise lead to bursting or leaking. For example, the base member 216, 316 can be made of metal such as steel or aluminum. It can be advantageous for the receptacle 218, 318 to be visible through the base member 216, 316 so that an operator of the system 200, 300 can view the receptacle 218, 318 during a fluid transfer procedure. Accordingly, the base member 216, 316 can alternatively be made of a substantially clear and strong plastic material.

The receptacle 218, 318 and the base member 216, 316, once engaged with one another, can be placed in a sterile bag 264, 364 before use for sterility purposes. The sterile bag 264, 364 can be formed by first 266, 366 and second 268, 368 panels sealed about portions of their perimeters. A first edge 270, 370 of the sterile bag 264, 364 can initially be unsealed and include an activatable adhesive strip 272, 372 to allow receipt of the receptacle 218, 318 and the base member 216, 316 and therefore seal the edge 270, 370. A second edge 274, 374 of the sterile bag 264, 364 can include a puncture zone 275, 375 to allow the extension line 224, 324 to engage with the second end 344 of the receptacle 218, 318. A corner 276, 376 of the sterile bag 264, 364 can include a peel off tab 278, 378 for decoupling the first panel 266, 366 and the second panel 268, 368 after a procedure has been completed to expose the receptacle 218, 318 and the base member 216, 316.

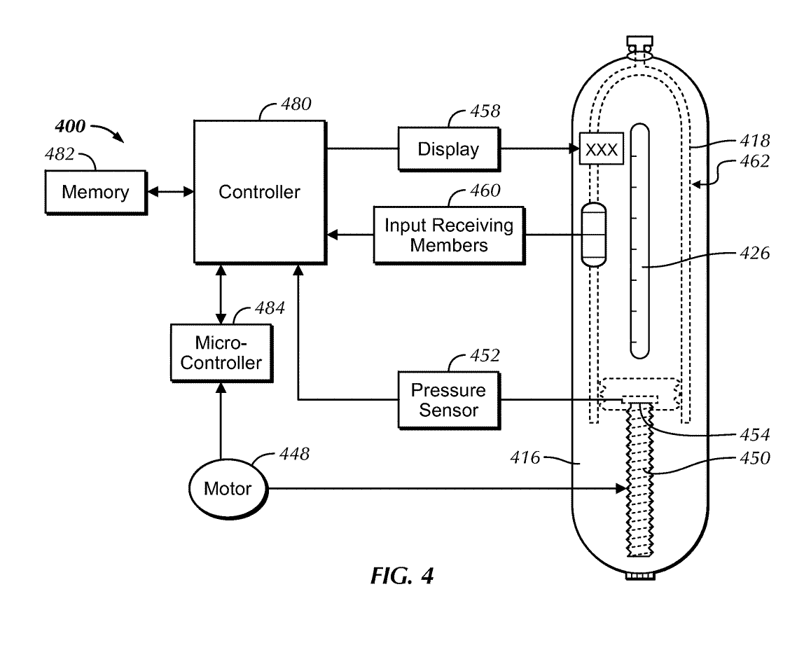

FIG. 4 illustrates a functional flow diagram of components included in a fluid delivery or removal system 400. A pressure sensor 452 can be incorporated within, or positioned on or near, a surface of a tip 454 of a piston 450 and configured to measure a load exerted thereon by fluid 426 within a receptacle 418 or the closed system. The pressure sensor 452 can be a strain beam type sensor, a load cell, a piezo-resistive transducer or another sensor type. An electrical signal output by the pressure sensor 452 can be input to a controller 480 where the signal can be digitally processed to derive and record electronic data representing, among other things, the magnitude and duration of applied fluid pressure to a dilatation balloon. The electronic data representing this information can be presented on a display 458 and recorded in a memory 482 for each inflation cycle.

The display 458 can be built into the base member 416 and provide a digital readout of fluid measurements or indications. For example, the digital readout can present fluid pressure readings, balloon size readings (e.g., diameter and/or volume) in the case of a dilatation balloon catheter, amount of fluid dispensed, amount of fluid remaining in the fluid delivery system 400, whether any of the readings are changing, error indications, timer readings (e.g., in the case a balloon dilatation catheter, how long the balloon has been inflated at a treatment site in the body), temperature readings, bar graphs that correspond to the readings, or any other desired measurement or reading depending on the particular application.

The display 458 can be configured with lights and sounds to grab an operator's attention. For example, the display 458 can glow in the dark or have appropriate buttons and lights for enabling the digital readout to light up. This may be desirable in the many laboratories that are kept in dark conditions. The display 458 can emit a sound when certain measurements are received from the pressure sensor 452 and processed by the controller 480. The display 458 can have one or more lights that indicate balloon inflation pressure or size. Using the example of a dilatation balloon, lights may indicate whether the balloon is increasing in pressure or size or decreasing in pressure or size.

One or more input receiving members 460, accessible on an outer surface of the base member 416 and in electrical communication with the controller 480, can power on and power off the system 400 or receive a desired pressure or other parameter set point from the operator and output it to the controller 480. The controller 480 can compare the sensed fluid pressure with the desired pressure set point, for example, and output start or stop instructions to a powered actuator 448, which in turn directs movement or non-movement of the piston 450. The controller 480 can be programmed to automatically react to changes in pressure according to data received from the pressure sensor 452. For example, an inputted maximum fluid pressure value may be set such that if the controller 480 receives information from the pressure sensor 452 that the pressure has reached the maximum pressure, it may slow down the powered actuator 448 or stop it completely in order not to exceed the maximum value. The one or more input receiving members 460 can optionally receive desired inflation or deflation speeds at which fluid is dispensed from or received by the receptacle 418. A microcontroller 484 can control the speed of the powered actuator 448, enabling the fluid to be delivered or received at a range of speeds.

The controller 480 can optionally be designed to permit the operator to select from a list of preprogrammed parameters, such as preprogrammed pressure-time curves or data previously recorded for a specific patient or patient grouping, via the one or more input receiving members 460. For example, the operator can select one of a variety of pressure-time curves that require a predetermined pressure to be reached within a predetermined amount of time. Preprogrammed options can provide for convenient and uniform operation of the base member 416 and receptacle 418 when inflating or deflating the dilatation balloon.

The controller 480 can also optionally be designed to calculate the volume of fluid 426 discharged. For example, the controller 480 can monitor the position of the piston tip 454 by counting the number of revolutions it has been turned by the powered actuator 448 and, based on this information, can calculate the volume of fluid 426 discharged. When the receptacle 418 is calculated to be empty, the controller 480 can instruct the powered actuator 448 to reverse completely to allow a ‘ready to use’ receptacle/seal member kit 462 to be inserted into the base member 416.

FIGS. 5A-5C illustrate example locking configuration between a surface cavity of a seal member 536 and a tip 554 of a piston 550. In various examples, the surface of the seal member 536 includes a cavity 586 into which the tip 554 can be placed. As shown in FIG. 5A, the cavity 586 can be threaded 588 to engage with mating threads 590 extending around the outer surface of tip 554. As shown in FIG. 5B, the cavity 586 can include one or more slots 592 sized and shaped to receive a projection 594 on the tip 554. Once the projection 594 is aligned with the slot 592, an operator can move the projection 594 through the slot 592 and then rotate the piston 550 so that the projection and slot are no longer aligned thereby preventing inadvertent disengagement. Or, as shown in FIG. 5C, the tip 554 can be press fit into the cavity 586 of the seal member 536. Optionally, the piston 550 can include a quick release mechanism that releases the engagement between the seal member 536 and its tip 554.

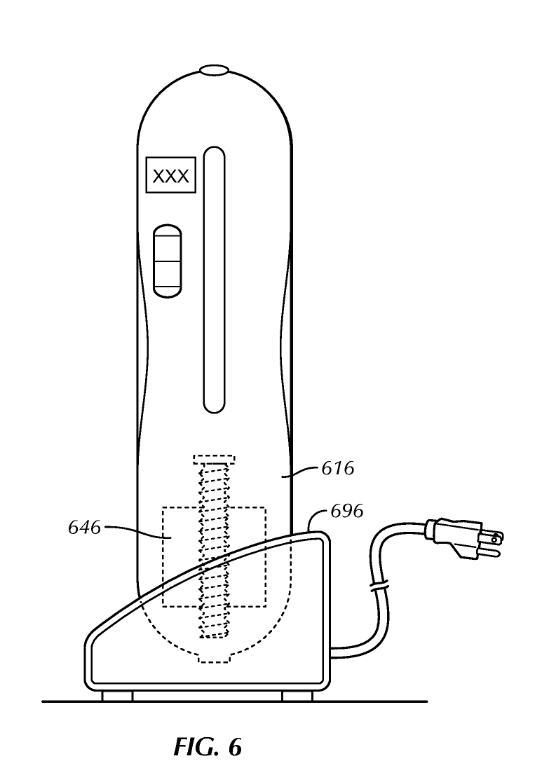

FIG. 6 illustrates an example engagement between a charging station 696 and a base member 616. The charging station 696 can be configured to transfer hardwired power from an outlet, for example, to a rechargeable power supply 646 housed by the base member 616. The base member 616 can be docked in the charging station 696 after each use or when a low battery power indication is signaled to an operator.

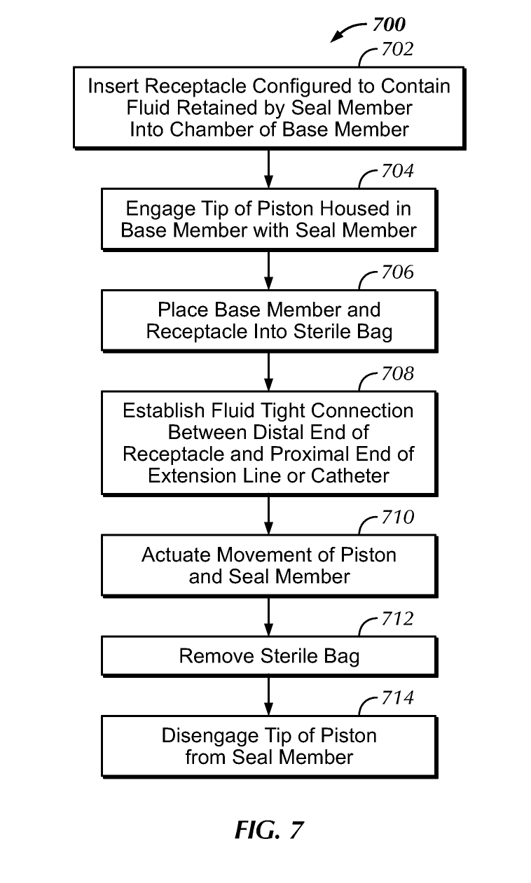

FIG. 7 illustrates a method 700 of using a fluid delivery or removal system to inflate or deflate a dilatation balloon of a catheter. At 702, a refillable or disposable receptacle configured to contain a volume of fluid partially retained by a seal member can be inserted into a chamber of a reusable base member. At 704, the tip of a piston housed in the base member can be engaged with the seal member with a pressure sensor positioned between a portion of the piston and a portion of the seal member. In an example, engagement of the piston tip and the seal member can include screwing the seal member onto the tip. In another example, engagement can include aligning a projection of the tip with a slot of the seal member, passing the projection through the slot, and rotating the seal member relative to the tip.

At 706, the base member and the receptacle can be placed into and sealed within a sterile bag. At 708, a fluid-tight connection between a dispensing opening of the receptacle and an end of a tubular body of the dilatation balloon or an extension line can be established to allow inflation fluid to move between the receptacle and the dilatation balloon. Establishing this connection can include puncturing a portion of the sterile bag near the receptacle's dispensing opening.

With the base member and the receptacle engaged with one another and positioned within a sterile environment, and with the contents of the receptacle in fluid communication with the dilatation balloon, the piston and the seal member can be caused to move in a distal or proximal direction within the receptacle at 710. This movement delivers fluid to, or removes fluid from, the dilatation balloon. A powered actuator can be used to move the piston and seal member based on a comparison of parameter set points established by an operator and measurements sensed or calculated using pressure sensor or power actuator data. Once a desired series of inflations and deflations has been completed and a lesion with a vessel is cleared or sufficiently opened, the dilatation balloon can be removed from the vessel.

At 712, the sterile bag can be removed from the base member and the receptacle and, at 714, the tip of the piston can be disengaged from the seal member allowing the receptacle and seal member to be removed from the base member. The base member can then be cleaned or a rechargeable battery within the base member can be charged.

Closing Notes:

The present fluid delivery or removal systems and methods allow for operator control with a single hand, leaving the second hand free to control the position of a catheter in a vessel or to perform other needed tasks. The system components are easy to use, precise in their measurements, able to deliver or remove fluid at a uniform rate, and inexpensive to maintain through use of a reusable base component and a refillable or disposable receptacle component. A pressure sensor attached to or incorporated within the base component allows the systems to connect to a dilatation balloon catheter or other expandable-type member, for example, and electronically measure, display, and record inflation or deflation data. These electronic features can increase the convenience and safe utilization of current and future medical procedures involving fluid delivery or removal.

The above Detailed Description includes references to the accompanying drawings, which form a part of the Detailed Description. The Detailed Description should be read with reference to the drawings. The drawings show, by way of illustration, specific embodiments in which the present systems and methods can be practiced. These embodiments are also referred to herein as “examples.”

The above Detailed Description is intended to be illustrative and not restrictive. For example, the above-described examples (or one or more features or components thereof) can be used in combination with each other. Other embodiments can be used, such as by one of ordinary skill in the art upon reviewing the above Detailed Description. Also, various features or components have been or can be grouped together to streamline the disclosure. This should not be interpreted as intending that an unclaimed disclosed feature is essential to any claim. Rather, inventive subject matter can lie in less than all features of a particular disclosed embodiment. Thus, the following claim examples are hereby incorporated into the Detailed Description, with each example standing on its own as a separate embodiment:

In Example 1, a fluid delivery or removal system can comprise a receptacle, a base member, and a pressure sensor. The receptacle can be configured to contain a volume of fluid and extend from a first end having an opening of a first diameter to a second end having an opening of a smaller, second diameter. A seal member can be positioned near the first end within the opening of the first diameter and engage an interior surface of the receptacle along its perimeter. The base member can include a piston and a powered actuator for imparting motive forces to the piston. A tip of the piston can be engageable with the seal member when the receptacle is placed in a chamber of the base member. The pressure sensor can be incorporated within, or positioned on or near, a surface of the tip and configured to measure a load exerted thereon by the fluid.

In Example 2, the system of Example 1 can optionally further comprise a display configured to present an indication representative of the load exerted by the fluid.

In Example 3, the system of any one of Examples 1 or 2 can optionally further comprise one or more input receiving members for setting a desired pressure set point or an operating speed at which fluid is dispensed from or received by the receptacle. The input receiving member can be accessible from outside the base member and in electrical communication with the powered actuator.

In Example 4, the system of Example 3 can optionally further comprise a controller to process one or more signals obtained by the pressure sensor, compare the signals to the desired pressure set point, and send one or more actuation signals to the powered actuator.

In Example 5, the system of any one or any combination of Examples 1-4 can optionally further comprise a rechargeable power supply located within the base member. The rechargeable power supply can energize the powered actuator.

In Example 6, the system of Example 5 can optionally further comprise a charging station engageable with the base member and configured to transfer external power to the rechargeable power supply.

In Example 7, the system of any one or any combination of Examples 1-6 can optionally further comprise an extension line having a first end engageable with the receptacle and having a second end engageable with a catheter.

In Example 8, the system of any one or any combination of Examples 1-7 can optionally be configured such that the seal member is displaceable within the receptacle through axial movement of the piston for altering the volume of fluid within the receptacle.

In Example 9, the system of any one or any combination of Examples 1-8 can optionally be configured such that a surface of the seal member includes a cavity, and the tip of the piston includes a projection sized and shaped to be received by the cavity.

In Example 10, the system of Example 9 can optionally be configured such that the cavity is configured to permit the projection to rotate in the seal member.

In Example 11, the system of Example 10 can optionally be configured such that the rotational movement of the projection within the cavity causes engagement between the tip and the seal member.

In Example 12, the system of any one or any combination of Examples 1-11 can optionally be configured such that the base member defines a chamber sized and shaped to receive a portion of the receptacle.

In Example 13, the system of Example 12 can optionally be configured such that the bae member surrounds a portion of the receptacle to restrain outward expansion of the receptacle during a fluid delivery procedure.

In Example 14, the system of any one of Examples 12 or 13 can optionally further comprise a sterile bag sized and shaped to receive the base member and the receptacle after the receptacle is positioned with the chamber of the base member. The sterile bag can be formed by first and second panels sealed about portions of their perimeters.

In Example 15, the system of Example 14 can optionally be configured such that an edge of the sterile bag includes a puncture zone to allow an extension line to engage with the second end of the receptacle after the receptacle has been placed within the sterile bag.

In Example 16, the system of any one of Examples 14 or 15 can optionally be configured such that an open edge of the sterile bag includes an adhesive to allow receipt of the receptacle and the base member and thereafter seal the open edge.

In Example 17, the system of any one or any combination of Examples 14-16 can optionally be configured such that a corner of the sterile bag includes a peel off tab for decoupling the first panel and the second panel and exposing the receptacle and the base member.

In Example 18, the system of any one or any combination of Examples 1-17 can optionally be configured such that the piston includes a threaded portion rotatably driven by the powered actuator.

In Example 19, the system of Example 18 can optionally be configured such that the threaded portion extends along a substantial portion of the piston.

In Example 20, the system of any one or any combination of Examples 1-19 can optionally be configured such that the powered actuator includes a screw motor that moves the piston along its axis in response to signals from one or more input receiving members.

In Example 21, the system of any one or any combination of Examples 1-20 can optionally be configured such that the receptacle and the seal member form a disposable component.

In Example 22, the system of Example 21 can optionally be configured such that the disposable component is in the form of a ‘ready to use’ kit, with the receptacle containing the volume of fluid.

In Example 23, a method can comprise inserting a receptacle configured to contain a volume of fluid retained in part by a seal member into a chamber of a base member. A tip of a piston included in the base member can be engaged with the seal member, with a pressure sensor positioned between a portion of the piston and a portion of the seal member. A fluid-tight pathway between a dispensing opening of the receptacle and a distal end of a catheter can be established, and a desired pressure set point can be inputted using one or more input receiving members accessible from outside of the base member. The desired pressure set point can cause the piston to advance or retract in an axial direction to move the seal member within the receptacle and fluid through the pathway. A pressure applied to fluid within the pathway can be sensed using the pressure sensor before, during, or after movement of the piston and seal member.

In Example 24, the method of Example 23 can optionally be configured such that engaging the tip of the piston with the seal member includes rotating the receptacle such that the seal member screws onto the tip.

In Example 25, the method of Example 23 can optionally be configured such that engaging the tip of the piston with the seal member includes rotating the receptacle such that at least one retaining flange on the tip engages a slot within the seal member.

In Example 26, the method of any one or any combination of Examples 23-25 can optionally be configured such that establishing the pathway between the dispensing opening of the receptacle and the distal end of the catheter includes engaging a first end of an extension line with the receptacle and engaging a second end of the extension line with the catheter.

In Example 27, the method of any one or any combination of Examples 23-26 can optionally be configured such that causing the piston to be advanced or retracted in the axial direction includes causing a powered actuator to impart a motive force to the piston.

In Example 28, the method of any one or any combination of Examples 23-27 can optionally be configured such that causing the piston to be advanced or retracted in the axial direction includes delivering fluid to a balloon of the catheter.

In Example 29, the method of any one or any combination of Examples 23-28 can optionally further comprise placing the base member and the receptacle in a sterile bag and sealing the sterile bag.

In Example 30, the method of Example 29 can optionally further comprise removing the sterile bag from the base member and the receptacle, and docking the base member in a charging station.

In Example 31, the method of Example 30 can optionally further comprise disengaging the tip of the piston from the seal member, removing the receptacle from the base member, and disposing of the receptacle.

In Example 32, the system or method of any one or any combination of Examples 1-31 can optionally be configured such that all components or options recited are available to use or select from.

Certain terms are used throughout this patent document to refer to particular features or components. As one skilled in the art will appreciate, different people may refer to the same feature or component by different names. This patent document does not intend to distinguish between components or features that differ in name but not in function.

For the following defined terms, certain definitions shall be applied unless a different definition is given elsewhere in this patent document.

a) The terms “a,” “an,” and “the” are used to include one or more than one, independent of any other instances or usages of “at least one” or “one or more.”

b) The term “or” is used to refer to a nonexclusive or, such that “A or B” includes “A but not B,” “B but not A,” and “A and B.”

c) All numeric values are assumed to be modified by the term “about,” whether or not explicitly indicated. The term “about” refers to a range of numbers that one of skill in the art considers equivalent to the recited value (e.g., having the same function or result). In many instances, the term “about” can include numbers that are rounded to the nearest significant figure.

d) The recitation of numerical ranges by endpoints includes all numbers and sub-ranges within and bounding that range (e.g., 1 to 4 includes 1, 1.5, 1.75, 2, 2.3, 2.6, 2.9, etc. and 1 to 1.5, 1 to 2, 1 to 3, 2 to 3.5, 2 to 4, 3 to 4, etc.).

e) The terms “patient” and “subject” are intended to include mammals, such as for human or veterinary applications.

f) The terms “distal” and “proximal” are used to refer to a position or direction relative to an operator. “Distal” and “distally” refer to a position that is distant from, or in a direction away from, the physician. “Proximal” and “proximally” refer to a position that is closer to, or in a direction toward, the physician. In the present systems and methods, a proximal portion of the base member can include the piston and a distal portion of the base member can include a chamber to receive the receptacle. Similarly, a proximal portion of the receptacle can include the seal member that engages with the distal tip of the piston and a distal portion of the receptacle can include an inlet/outlet portion for fluid transfer.

The scope of the present systems and methods should be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended; that is, a system or method that includes features or components in addition to those listed after such a term in a claim are still deemed to fall within the scope of that claim. Moreover, in the following claims, the terms “first,” “second” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

What is claimed is:

1. A fluid delivery or removal system, comprising:
a receptacle configured to contain a volume of fluid, the receptacle having a first end having an opening of a first diameter and a second end having an opening of a second diameter that is smaller than the first diameter;
a seal member positioned near the first end and engaged with an interior surface of the receptacle along its perimeter;
a base member including a piston and a powered actuator for imparting motive forces to the piston, the piston having a tip engageable with the seal member; and
a pressure sensor incorporated within, or positioned on, a surface of the tip and configured to measure a load exerted thereon by the fluid.

2. The system of claim 1, further comprising a display configured to present an indication representative of the load exerted by the fluid.

3. The system of claim 1, further comprising one or more input receiving members, accessible from outside the base member and in electrical communication with the powered actuator, for setting a desired pressure set point or an operating speed at which fluid is dispensed from or received by the receptacle.

4. The system of claim 3, further comprising a controller to process one or more signals obtained by the pressure sensor, compare the signals to the desired pressure set point, and send one or more actuation signals to the powered actuator.

5. The system of claim 1, further comprising a rechargeable power supply located within the base member, wherein the powered actuator is energized by the rechargeable power supply.

6. The system of claim 5, further comprising a charging station engageable with the base member and configured to transfer external power to the rechargeable power supply.

7. The system of claim 1, wherein a surface of the seal member includes a cavity, and the tip of the piston includes a projection sized and shaped to be received by the cavity.

8. The system of claim 7, wherein the cavity is configured to permit the projection to rotate in the seal member.

9. The system of claim 8, wherein the rotational movement of the projection within the cavity causes engagement between the tip and the seal member.

10. The system of claim 1, wherein the base member defines a chamber sized and shaped to receive a portion of the receptacle.

11. The system of claim 10, wherein the base member surrounds a portion of the receptacle to restrain outward expansion of the receptacle during a fluid delivery procedure.

12. The system of claim 10, further comprising a sterile bag sized and shaped to receive the base member and the receptacle, after the receptacle is positioned within the chamber of the base member, and formed by first and second panels sealed about portions of their perimeters.

13. The system of claim 12, wherein an edge of the sterile bag includes a puncture zone to allow an extension line to engage with the second end of the receptacle after the receptacle has been placed within the sterile bag.

14. The system of claim 12, wherein an open edge of the sterile bag includes an adhesive to allow receipt of the receptacle and the base member and thereafter seal the open edge.

15. The system of claim 12, wherein a corner of the sterile bag includes a peel off tab for decoupling the first panel and the second panel and exposing the receptacle and the base member.

16. The system of claim 1, wherein the piston includes a threaded portion rotatably driven by the powered actuator.

17. The system of claim 1, wherein the powered actuator includes a screw motor that moves the piston along its axis in response to signals from one or more input receiving members.

18. A method, comprising:
inserting a receptacle configured to contain a volume of fluid retained in part by a seal member into a chamber of a base member;
engaging a tip of a piston with the seal member, including positioning a pressure sensor between a portion of the piston and a portion of the seal member;
establishing a fluid-tight pathway between a dispensing opening of the receptacle and a distal end of a catheter;
inputting a desired pressure set point using one or more input receiving members accessible from outside of the base member, including causing the piston to advance or retract in an axial direction to move the seal member within the receptacle and deliver fluid to, or remove fluid from, the catheter through the pathway; and
sensing a pressure applied to fluid within the pathway.

19. The method of claim 18, wherein engaging the tip of the piston with the seal member includes rotating the receptacle such that the seal member screws onto the tip.

20. The method of claim 18, wherein engaging the tip of the piston with the seal member includes rotating the receptacle such that at least one retaining flange on the tip engages a slot within the seal member.

21. The method of claim 18, wherein establishing the pathway between the dispensing opening of the receptacle and the distal end of the catheter includes engaging a first end of an extension line with the receptacle and engaging a second end of the extension line with the catheter.

22. The method of claim 18, wherein causing the piston to advance or retract in the axial direction includes causing a powered actuator to impart a motive force to the piston.

23. The method of claim 18, further comprising placing the base member and the receptacle in a sterile bag and sealing the sterile bag.

24. The method of claim 23, further comprising removing the sterile bag from the base member and the receptacle, and docking the base member in a charging station.

25. The method of claim 24, further comprising disengaging the tip of the piston from the seal member, removing the receptacle from the base member, and disposing of the receptacle.
